(19) United States
(12) Patent Application Publication
(10) Pub. No.: US 2025/0090685 A1
(43) Pub. Date: Mar. 20, 2025

(54) Cationic Hyperbranched Starch-based Gene Carrier, Preparation Method and Application Thereof

(71) Applicant: Jiangnan University, Wuxi (CN)

(72) Inventors: Li CHENG, Wuxi (CN); Yue Sun, Wuxi (CN); Zhengbiao Gu, Wuxi (CN); Yan Hong, Wuxi (CN); Zhaofeng Li, Wuxi (CN); Caiming Li, Wuxi (CN); Xiaofeng Ban, Wuxi (CN)

(21) Appl. No.: 18/963,609

(22) Filed: Nov. 28, 2024

Related U.S. Application Data
(63) Continuation of application No. PCT/CN2022/103778, filed on Jul. 5, 2022.

(30) Foreign Application Priority Data
May 30, 2022 (CN) 202210601822X

(51) Int. Cl. A61K 48/00; C12N 15/113; C12P 19/04; C12P 19/18

(52) U.S. Cl. CPC A61K 48/0041; C12N 15/113; C12P 19/04; C12P 19/18; C12N 2310/14

(57) ABSTRACT

The disclosure discloses a method for preparing a nanoscale cationic hyperbranched starch-based gene carrier, which mainly includes the following steps: heating and gelatinizing a dextrin solution, then obtaining a highly branched cluster dextrin molecule having abundant short chains through hydrolysis and transglycosylation of starch branching enzymes, and then performing an etherification reaction to obtain cationic polymers with different degrees of substitution. The polymer is controllable in degradation, the highly branched structure thereof can reduce the requirement of the gene carrier on the high degree of substitution of cationic starch to a certain extent, and the cytotoxicity is obviously reduced. In addition, the polymer carrier can form a stable nanocomplex with a gene fragment, and has wide application in gene therapy as an efficient gene carrier.

### TECHNICAL FIELD

The disclosure relates to a cationic hyperbranched starch-based gene carrier, a preparation method and application thereof, and belongs to the field of medicines.

### BACKGROUND

The composition of carriers of gene-based drugs is complex, and it is difficult to find the cause of side effects. The occurrence of a certain proportion and varying degrees of allergic reactions after vaccine injection greatly reduces people's trust in gene drugs. At present, the main carriers of gene-based drugs are nanoliposomes. Polyethylene glycol (PEG) is mainly used as a water carrier and stabilizer to increase the stability and shelf life of vaccines. PEG is a polyether compound widely used in pharmaceuticals, cosmetics and food additives. It has been reported that there are few people who are allergic to PEG. However, research results indicate that during the process of PEGylation, the conformation and/or chemical structure of PEG covalently attached to the surface of lipid nanoparticles may undergo changes, which may alter and increase the structure of PEG, and make it have a certain degree of allergenicity. Therefore, it is crucial to find a safe and effective gene carrier with simple structure.

Among numerous gene delivery carriers, polyamide-amine (PAMAM), as one of the most extensively and deeply studied polymers in the family of dendrimers, has attracted widespread attention from researchers due to its unique structure and properties. PAMAM is a highly branched polymer macromolecule with a clear structure and uniform size, mainly composed of three parts: core, internal (branch) and shell (terminal group), where ethylenediamine serves as the core, methyl acrylate and ethylenediamine are grafted in sequence, and amino or carboxyl groups serve as the terminal functional groups. Such hyperbranched polymer has a large number of terminal functional groups, good solubility, low molecular entanglement, high geometric symmetry in structure, artificially controllable molecular weight and structure, good gene loading effects and high transfection efficiency. In the last decade, PAMAM has been widely used in biomedicine, catalyst carriers, membrane materials and other fields due to its novel structure and unique properties, especially in the delivery of drugs and genes in the biomedical field.

As an artificially synthesized cationic gene carrier, PAMAM exhibits increasingly good gene loading and protective effects with increasing synthesis generations. Research has found that when gene fragments interact with PAMAM macromolecules, the gene fragments are often distributed in internal gaps of the dendritic structure of the PAMAM macromolecules. This molecular encapsulation mechanism can effectively improve the water solubility and controlled release of drugs, and the geometric shapes of the carriers and the gene fragments remain almost unchanged. Starch, as one of the few natural macromolecules with high branching structures in nature, is one of the most widely used drug carriers, and is often used as a disintegrant, a diluent, and a binder in the form of starch paste during wet granulation processes. However, natural starch cannot meet the requirements as a gene carrier due to poor hydrophobicity and low resistance, and needs to be modified by certain physical and chemical means to meet the demand as an ideal gene carrier. In modification processes of starch, enzymatic hydrolysis or acid hydrolysis can significantly reduce the molecular weight, but the molecular weight distribution of the product is uneven, and the natural branching structure is damaged to varying degrees.

### SUMMARY

The disclosure provides a method for preparing a highly branched starch-based cationic polymer gene carrier having a low degree of substitution and application thereof. The preparation method is simple in process, easy to control and low in cost.

A method for preparing a cationic hyperbranched starch-based gene carrier provided by the disclosure includes the following steps: increasing the degree of branching of a starch molecule, reducing the molecular weight of the starch molecule, and providing certain resistance for a system through transglycosylation and hydrolysis of starch branching enzymes, and then preparing a cationic branched starch-based gene carrier through an etherification reaction to successfully graft a cationic group onto the carrier, thereby achieving effective delivery of siRNA. After enzymatic hydrolysis, the starch has an increased degree of branching and a decreased molecular weight, the particle size distribution of a prepared complex can reach the nanoscale, thus the complex possesses the advantages of nanoscale carriers, and a high specific surface area of the highly branched structure increases the loading capacity of siRNA through adsorption.

The cationic hyperbranched starch-based gene carrier prepared by the method of the disclosure has a low degree of substitution and can achieve complete encapsulation of siRNA. With the increase in the degree of substitution, the encapsulation effect is better. With the increase in the degree of branching of the starch-based carrier, the complex formed is more uniform, and the minimum size can reach about 300 nm, belonging to the range of nanoscale delivery drugs.

The starch branching enzymes can increase the number of non-reducing ends of the starch molecule, produce many branched short chains, and achieve artificial regulation of the starch structure, thereby obtaining a natural biomacromolecule with a highly branched structure. The halogen or epoxy group in an etherifying agent can undergo an etherification reaction with a hydroxyl group in the starch molecule, producing a positively charged starch ether derivative. The positively charged starch derivative can interact with a gene fragment electrostatically to form a stable complex. The cationic hyperbranched starch-based carrier with a branched structure obtained in the disclosure has strong ability to encapsulate and protect gene fragments. By comparing gene carriers having different degrees of branching and degrees of substitution, the performance of the cationic hyperbranched starch-based carrier is tested, which is of great significance for developing new safe and non-toxic gene carriers and expanding utilization of starch resources.

The technical solutions of the disclosure are as follows:

A method for preparing a cationic hyperbranched starch-based gene carrier includes: using starch/dextrin treated with starch branching enzymes as a substrate, and chemically modifying the substrate with a cationic etherifying agent to obtain a cationic branched starch-based gene carrier, where the content of α-1,6 bonds in the cationic hyperbranched starch-based gene carrier is 5%-11%; and the degree of substitution of the cationic hyperbranched starch-based gene carrier is 0.030-0.080.

In one implementation of the disclosure, the cationic hyperbranched starch-based gene carrier forms a 300-400 nm nanocomplex with a gene drug.

In one implementation of the disclosure, specific steps for preparing the cationic hyperbranched starch-based gene carrier are as follows:
(1) preparation of substrate
mixing starch/dextrin with distilled water to prepare a water solution, preserving heat in a water bath for gelatinization, stirring and adding starch branching enzymes, performing gelatinizing and enzyme deactivation, and performing freeze-drying, grinding and screening to obtain the substrate, i.e., the starch/dextrin treated with the starch branching enzymes; and
(2) preparation of cationic hyperbranched starch-based gene carrier by substrate modification
dispersing the substrate obtained in step (1) in anhydrous ethanol to form a mixture; adjusting the pH of the cationic etherifying agent to 9-10; then adding the cationic etherifying agent to the mixture for reaction by heating; performing cooling to room temperature after the reaction to produce a yellow or light yellow primary product; adding glacial acetic acid for neutralizing the system to a neutral pH; and then performing filtering, washing and drying to obtain the cationic hyperbranched starch-based gene carrier.

In one implementation of the disclosure, the cationic etherifying agent is a solution of trimethylammonium chloride containing 3-chloro-2-hydroxypropyl.

In one implementation of the disclosure, the starch treated with the starch branching enzymes is branched starch (denoted as RG-S).

In one implementation of the disclosure, the dextrin treated with the starch branching enzymes is branched dextrin (denoted as RG-M).

In one implementation of the disclosure, specific steps for preparing the cationic hyperbranched starch-based gene carrier using starch as the substrate are as follows:
(1) preparation of branched starch (RG-S)
mixing starch with distilled water to prepare a water solution of starch, preserving heat in a water bath for gelatinization, stirring and adding starch branching enzymes, performing gelatinizing and enzyme deactivation, and performing freeze-drying, grinding and screening to obtain the substrate, i.e., the starch treated with the starch branching enzymes, denoted as branched starch (RG-S); and
(2) preparation of cationic hyperbranched starch-based gene carrier by branched starch (RG-S) modification
dispersing the RG-S obtained in step (1) in anhydrous ethanol to form a mixture; adjusting the pH of an etherifying agent, i.e. a (3-chloro-2-hydroxypropyl) trimethylammonium chloride solution to 9-10 for reaction by heating; performing cooling to room temperature after the reaction to produce a yellow or light yellow cationic starch primary product; adding glacial acetic acid to the cationic starch primary product for neutralizing the reaction system to a pH value of 7; washing the cationic starch primary product thoroughly with anhydrous ethanol through vacuum suction filtration until there is no silver chloride precipitation when silver nitrate is added dropwise to the filtrate; and performing suction filtration and drying in a 37° C. oven to a constant weight to obtain a cationic starch gene carrier, denoted as C-RG-S.

In one implementation of the disclosure, in step (1), the concentration of the water solution of starch is 10%-30% (w/v) on a dry basis.

In one implementation of the disclosure, in step (1), the starch branching enzymes are 1,4-α-glucan branching enzyme from Rhodothermus obamensis (Ro-GBE) and a 1,4-α-glucan branching enzyme from Geobacillus thermoglucosidans (Gt-GBE).

In one implementation of the disclosure, in step (1), the starch branching enzyme Gt-GBE has a reaction temperature of 50° C.-60° C., an enzyme concentration of 25-35 U/g, and a reaction time of 10-15 h.

In one implementation of the disclosure, in step (1), the starch branching enzyme Ro-GBE has a reaction temperature of 55° C.-65° C., an enzyme concentration of 30-40 U/g, and a reaction time of 8-12 h.

In one implementation of the disclosure, in step (1), the step of adding starch branching enzymes is to add 35 U/g of Ro-GBE and 30 U/g of Gt-GBE for enzymatic hydrolysis for 10 h.

In one implementation of the disclosure, in step (2), the molar ratio of dehydrated glucose units in the branched starch (RG-S) to NaOH to CTA is (1-1.2):(1-1.2):(1-1.5), the water content of the mixed system is not higher than 10%, the reaction temperature is 50-70° C., and the reaction time is 1-4 h.

In one implementation of the disclosure, specific steps for preparing the cationic hyperbranched starch-based gene carrier using dextrin as the substrate are as follows:
(1) preparation of branched dextrin (RG-M)
mixing dextrin with distilled water to prepare a water solution, preserving heat in a water bath for gelatinization, stirring and adding starch branching enzymes, performing gelatinizing and enzyme deactivation, and performing freeze-drying, grinding and screening to obtain the substrate, i.e., the dextrin treated with the starch branching enzymes, denoted as RG-M; and
(2) preparation of cationic hyperbranched starch-based gene carrier by branched dextrin (RG-M) modification
dispersing the RG-M obtained in step (1) in anhydrous ethanol to form a mixture; adjusting the pH of an etherifying agent, i.e. a (3-chloro-2-hydroxypropyl) trimethylammonium chloride solution to 9-10 for reaction by heating; performing cooling to room temperature after the reaction to produce a yellow or light yellow cationic dextrin primary product; adding glacial acetic acid to the cationic dextrin primary product for neutralizing the reaction system to a pH value of 7; washing the cationic dextrin primary product thoroughly with anhydrous ethanol through vacuum suction filtration until there is no silver chloride precipitation when silver nitrate is added dropwise to the filtrate; and performing suction filtration and drying in a 37° C. oven to a constant weight to obtain a cationic dextrin gene carrier, denoted as C-RG-M.

In one implementation of the disclosure, in step (1), the concentration of the water solution of dextrin is 10%-30% (w/v) on a dry basis.

In one implementation of the disclosure, in step (1), the starch branching enzymes are starch branching enzymes Ro-GBE and Gt-GBE from Rhodothermus obamensis and Geobacillus thermoglucosidans.

In one implementation of the disclosure, in step (1), the step of adding starch branching enzymes is to firstly add the starch branching enzyme Gt-GBE and then add the starch branching enzyme Ro-GBE.

In one implementation of the disclosure, in step (1), the starch branching enzyme Gt-GBE has a reaction temperature of 50° C.-60° C., an enzyme concentration of 25-35 U/g, and a reaction time of 10-15 h.

In one implementation of the disclosure, in step (1), the starch branching enzyme Ro-GBE has a reaction temperature of 55° C.-65° C., an enzyme concentration of 30-40 U/g, and a reaction time of 8-12 h.

In one implementation of the disclosure, in step (2), the molar ratio of dehydrated glucose units in the branched dextrin to NaOH to CTA is (1-1.2):(1-1.2):(1-1.5), the water content of the mixed system is not higher than 10%, the reaction temperature is 50-70° C., and the reaction time is 1-4 h.

In one implementation of the disclosure, the prepared cationic hyperbranched starch-based gene carrier is put into a dialysis bag, dialyzed in ultrapure water for 48-72 h, and then freeze-dried.

The disclosure obtains a cationic hyperbranched starch-based gene carrier by the aforementioned method.

The second objective of the disclosure is to provide application of the cationic hyperbranched starch-based gene carrier in preparation of gene drugs.

In one implementation of the disclosure, the application is to provide application of the cationic hyperbranched starch-based gene carrier as a non-viral gene carrier in gene therapy.

In one implementation of the disclosure, the cationic hyperbranched dextrin gene carrier with a low degree of substitution and loaded with a gene drug is obtained by loading the cationic hyperbranched starch-based gene carrier with the gene drug through electrostatic attraction.

In one implementation of the disclosure, the gene drug is DNA or RNA.

In one implementation of the disclosure, the application is to mix the cationic hyperbranched starch-based gene carrier with a gene fragment at an N/P ratio of 2.0-3.5, the two can self-organize into a nanocomplex through electrostatic interaction, and thus the gene fragment is well loaded.

### Beneficial Effects

The cationic hyperbranched starch-based gene carrier prepared in the disclosure can load a large amount of gene drugs and has good hydrophobic drug carrying and gene drug binding abilities.

The cationic hyperbranched starch-based gene carrier prepared in the disclosure has a low degree of substitution and controllable reaction.

The complex formed by the cationic hyperbranched starch-based gene carrier prepared in the disclosure and siRNA has a positively charged surface.

The complex formed by the cationic hyperbranched starch-based gene carrier prepared in the disclosure and siRNA has a nanoscale particle size distribution.

### BRIEF DESCRIPTION OF FIGURES

FIG. 1 shows electropherograms illustrating the protection and leakage of siRNA with nano-delivery systems formed by cationic hyperbranched starch-based gene carriers in Examples 8-14, having different degrees of substitution and degrees of branching, and siRNA at different N/P ratios (lanes from left to right are respectively: bare siRNA, N/P=0.5, 1.0, 1.5, 2.0, 2.5, 3.0, 3.5).

FIG. 2 shows electropherograms illustrating the protection and leakage of siRNA with a nano-delivery system formed by a cationic hyperbranched starch-based gene carrier C-RG-M-4 with DS=0.077 in Example 17 and siRNA at the optimal N/P ratio (N/P=2.0) (lanes from left to right are respectively: bare siRNA and N/P=2.0) at 4 h, 24 h, 3 d and 7 d.

FIG. 3 shows SAXS curves of samples Y-M. Gt-M. Ro-M, and RG-M obtained in Test 4.

### DETAILED DESCRIPTION

The disclosure will be further explained in conjunction with specific examples, but the scope of protection of the disclosure is not limited thereto.

In the following examples, the corn starch was purchased from Shandong Shouguang Co., Ltd.; the maltodextrin was purchased from Shandong Baolingbao Biology Co., Ltd., China; and the two starch branching enzymes (EC 2.4.1.18) from Rhodothermus obamensis and Geobacillus thermoglucosidans are both from the laboratory of the inventor.

### Example 1

### Preparation of Cationic Hyperbranched Starch-Based Gene Carrier (C-RG-S-1)

(1) 10 g of common corn starch (on a dry basis) was weighed, prepared into a 10% (w/v) water solution of starch with distilled water, and gelatinized for 30 min.

(2) The gelatinized starch slurry was placed in a four-necked flask, preserved at 60° C. in a water bath for 15 min, stirred and added with 35 U/g of Ro-GBE and 30 U/g of Gt-GBE for reaction for 10 h, gelatinized and subjected to enzyme deactivation for 30 min, freeze-dried, ground and screened to obtain modified starch, denoted as RG-S.

(3) 5 g of the RG-S sample (on a dry basis) was weighed and dispersed in anhydrous ethanol to form a 5% (w/v) starch-ethanol mixture.

(4) An etherifying agent (3-chloro-2-hydroxypropyl) trimethylammonium chloride solution (CTA) was adjusted to a pH value of 10 with 10 mol/L of NaOH solution, and added to the starch-ethanol mixed solution, where the molar ratio of dehydrated glucose units in the starch to the NaOH to the CTA was 1:1:1, and the water content of the mixed system was not higher than 10%.

(5) The reaction was performed at 60° C. for 1 h, and cooling was performed to room temperature to produce a yellow or light yellow cationic branched starch primary product.

(6) Glacial acetic acid was added to the primary product for neutralizing the reaction system to a pH value of 7, the cationic branched starch primary product was washed thoroughly with anhydrous ethanol through vacuum suction filtration until there was no silver chloride precipitation when silver nitrate was added dropwise to the filtrate, and a suction filtration product was dried in a 37° C. oven to a constant weight.

(7) The product was loaded into a dialysis bag with a molecular weight cutoff of 1000 and dialyzed in ultrapure water for 72 h to obtain the product cationic hyperbranched starch-based gene carrier (denoted as C-RG-S-1).

### Example 2

### Preparation of Cationic Branched Starch C-RG-S-4

Steps (1) to (4) were the same as Example 1.

Referring to Example 1, step (5) was changed to: the reaction was performed at 60° C. for 4 h, and cooling was performed to room temperature to produce a yellow or light yellow cationic branched starch primary product.

Steps (6) to (7) were the same as Example 1, and the product cationic hyperbranched starch-based gene carrier (denoted as C-RG-S-4) was obtained.

### Example 3

### Preparation of Cationic Branched Dextrin C-Y-M-4

(1) 10 g of DE7-9 maltodextrin (on a dry basis) was weighed, prepared into a 10% (w/v) water solution of dextrin with distilled water, and gelatinized for 30 min.

(2) The gelatinized dextrin was freeze-dried, ground and screened to obtain modified dextrin, denoted as Y-M.

(3) 5 g of the Y-M sample (on a dry basis) was weighed and dispersed in anhydrous ethanol to form a 5% (w/v) dextrin-ethanol mixture.

(4) An etherifying agent (3-chloro-2-hydroxypropyl) trimethylammonium chloride solution (CTA) was adjusted to a pH value of 10 with 10 mol/L of NaOH solution, and added to the dextrin-ethanol mixed solution, where the molar ratio of dehydrated glucose units in the dextrin to the NaOH to the CTA was 1:1:1, and the water content of the mixed system was not higher than 10%.

(5) The reaction was performed at 60° C. for 4 h, and cooling was performed to room temperature to produce a yellow or light yellow cationic branched dextrin primary product.

(6) Glacial acetic acid was added to the primary product for neutralizing the reaction system to a pH value of 7, the cationic branched dextrin primary product was washed thoroughly with anhydrous ethanol through vacuum suction filtration until there was no silver chloride precipitation when silver nitrate was added dropwise to the filtrate, and a suction filtration product was dried in a 37° C. oven to a constant weight.

(7) The product was loaded into a dialysis bag with a molecular weight cutoff of 1000 and dialyzed in ultrapure water for 72 h to obtain the cationic hyperbranched starch-based gene carrier (denoted as C-Y-M-4).

### Example 4

### Preparation of Cationic Branched Dextrin C-Gt-M-4

Step (1) was the same as Example 3.

Referring to Example 3, step (2) was changed to: the gelatinized dextrin solution was placed in a four-necked flask, preserved at 55° C. in a water bath for 15 min, stirred and added with 30 U/g of Gt-GBE for reaction for 10 h, gelatinized and subjected to enzyme deactivation for 30 min, freeze-dried, ground and screened to obtain modified dextrin, denoted as Gt-M.

Steps (3) to (7) were the same as Example 3, and the product cationic hyperbranched starch-based gene carrier (denoted as C-Gt-M-4) was obtained.

### Example 5

### Preparation of Cationic Branched Dextrin C-Ro-M-4

Step (1) was the same as Example 3.

Referring to Example 3, step (2) was changed to: the gelatinized dextrin solution was placed in a four-necked flask, preserved at 60° C. in a water bath for 15 min, stirred and added with 35 U/g of Ro-GBE for reaction for 10 h, gelatinized and subjected to enzyme deactivation for 30 min, freeze-dried, ground and screened to obtain modified dextrin, denoted as Ro-M.

Steps (3) to (7) were the same as Example 3, and the product cationic hyperbranched starch-based gene carrier (denoted as C-Ro-M-4) was obtained.

### Example 6

### Preparation of Cationic Branched Dextrin C-RG-M-1

Step (1) was the same as Example 3.

Referring to Example 3, step (2) was changed to: the gelatinized dextrin solution was placed in a four-necked flask, preserved at 60° C. in a water bath for 15 min, stirred and added with 35 U/g of Ro-GBE and 30 U/g of Gt-GBE for reaction for 10 h, gelatinized and subjected to enzyme deactivation for 30 min, freeze-dried, ground and screened to obtain modified dextrin, denoted as RG-M.

Step (4) was the same as Example 3.

Step (5) was changed to: the reaction was performed at 60° C. for 1 h, and cooling was performed to room temperature to produce a yellow or light yellow cationic branched dextrin primary product.

Steps (6) to (7) were the same as Example 3, and the product cationic hyperbranched starch-based gene carrier (denoted as C-RG-M-1) was obtained.

### Example 7

### Preparation of Cationic Branched Dextrin C-RG-M-4

Step (1) was the same as Example 3.

Referring to Example 3, step (2) was changed to: the gelatinized dextrin solution was placed in a four-necked flask, preserved at 60° C. in a water bath for 15 min, stirred and added with 35 U/g of Ro-GBE and 30 U/g of Gt-GBE for reaction for 10 h, gelatinized and subjected to enzyme deactivation for 30 min, freeze-dried, ground and screened to obtain modified dextrin, denoted as RG-M.

Steps (3) to (7) were the same as Example 3, and the product cationic hyperbranched starch-based gene carrier (denoted as C-RG-M-4) was obtained.

### Test 1: Determination of Relative Content of α-1,6-Glycosidic Bonds

Samples were dissolved in heavy water (D₂O) to form starch milk with a concentration of 40 mg/mL, and gelatinized in boiling water for 30 min. The gelatinized samples were freeze-dried and dissolved again in D₂₀, and measured by 1H NMR (1H nuclear magnetic resonance). The relative content of α-1,6-glycosidic bonds can be obtained by calculating peak areas of absorption peaks corresponding to the α-1,4-glycosidic bond at 5.37 ppm and the α-1,6-glycosidic bond at 4.96 ppm in the spectrum.

Table 1 shows the content of α-1,6 glycosidic bonds in the cationic starch-based gene carriers having different degrees of branching prepared by the methods in the aforementioned examples.

10 g of common corn starch (on a dry basis) was weighed, prepared into a 10% (w/v) water solution of starch with distilled water, and gelatinized for 30 min. The gelatinized starch was freeze-dried, ground and screened to obtain modified starch, denoted as Y-S.

TABLE 1
Content of α-1,6 glycosidic bonds in starch-based
carriers having different degrees of branching

| Sample | Y-S | RG-S | Y-M | Gt-M | Ro-M | RG-M |
|---|---|---|---|---|---|---|
| Proportion of | 4.31% | 11.04% | 5.79% | 6.29% | 8.47% | 10.16% |
| α-1,6 bonds | | | | | | |

The starch branching enzymes Gt-GBE and Ro-GBE can increase the degree of branching of dextrin to a certain extent. Comparing the transglycosylation effect of the branching enzymes on different substrates, it was found that the proportion of α-1,6-glycosidic bonds in corn starch increased by 156%, and the proportion of α-1,6-glycosidic bonds in maltodextrin after enzymatic hydrolysis increased by 80%, indicating that the Gt-GBE and the Ro-GBE had better transglycosylation effect on starch than on dextrin. Comparing the transglycosylation effect of different branching enzymes on dextrin, it was found that the proportion of α-1,6 bonds in dextrin treated with the Gt-GBE and the Ro-GBE was significantly higher than that of a product treated with a single enzyme, indicating that the two branching enzymes have a synergistic effect when working together, and can significantly improve the degree of branching of a starch-based carrier.

### Test 2: Determination of Chain Length Distribution

10 mg of a sample was weighed and dissolved in 2 mL of a sodium acetate buffer (50 mM, pH 3.5), preheated at 37° C. for 15 min, added with 100 μL of isoamylase (10000 U/mL), deprotonated in a constant temperature water bath shaker (160 r/min) for 24 h, subjected to enzyme deactivation in a boiling water bath for 30 min, and centrifuged at 10000 r/min for 10 min. The supernatant was diluted and allowed to pass through a 0.22 μm water filter membrane. The chain length distribution of the sample was determined by HPAEC-PAD.

Table 2 shows the chain length distribution of the cationic starch-based gene carriers having different degrees of branching prepared by the methods in the aforementioned examples.

TABLE 2
Chain length distribution of starch-based carriers
having different degrees of branching

| | Chain length distribution percentage (%) | | | |
|---|---|---|---|---|
| Sample | DP 3-12 | DP 13-24 | DP 25-36 | DP >36 |
| Y-S | 8.77 ± 0.52 | 50.71 ± 0.34 | 23.96 ± 0.67 | 9.90 ± 0.37 |
| RG-S | 47.32 ± 0.27 | 17.22 ± 0.15 | 19.39 ± 0.08 | 6.90 ± 0.12 |
| Y-M | 49.98 ± 0.65 | 32.38 ± 0.52 | 8.37 ± 0.37 | 3.72 ± 0.75 |
| Gt-M | 51.15 ± 0.52 | 35.27 ± 0.20 | 9.84 ± 0.20 | 2.96 ± 0.04 |
| Ro-M | 55.57 ± 0.17 | 34.72 ± 0.01 | 8.21 ± 0.01 | 1.92 ± 0.01 |
| RG-M | 59.07 ± 0.11 | 29.87 ± 0.24 | 4.99 ± 0.17 | 1.11 ± 0.06 |

The starch branching enzymes Gt-GBE and Ro-GBE can catalyze hydrolysis of α-1,4-glycosidic bonds in starch molecules to a certain extent, causing long-chain segments with DP>13 to break and produce short chains with non-reducing ends. Maltodextrin is a product of acid hydrolysis or enzymatic hydrolysis of starch, and the original structure of starch has been partially damaged. The short chain content of dextrin not treated with any branching enzyme was significantly higher than that of starch treated with the Gt-GBE and the Ro-GBE. Comparing the hydrolysis effect of different branching enzymes on dextrin, it was found that the short chain content of dextrin treated with the Gt-GBE and the Ro-GBE was significantly higher than that of a product treated with a single enzyme, indicating that the two branching enzymes have a synergistic effect when working together and can significantly increase the short chain content of the starch-based carrier.

### Test 3: Determination of Molecular Weight Distribution

10 mg of a sample was weighed and dissolved in 2 mL of deionized water, placed in a boiling water bath for 30 min, and allowed to pass through a 0.22 μm water filter membrane. The molecular weight distribution of the sample was determined by Waters1525 high-performance liquid chromatograph, where the mobile phase was 0.1 M NaNO₃ and the chromatographic column was Ultrahydrogel™ Linear 300 mm×7.8 mmid×2.

Table 3 shows the molecular weight distribution of the starch-based gene carriers having different degrees of branching prepared by the methods in the aforementioned examples.

TABLE 3
Molecular weight distribution of starch-based carriers
having different degrees of branching

| Sample | | Y-M | Gt-M | Ro-M | RG-M |
|---|---|---|---|---|---|
| Molecular weight | Peak1 | 85524 | 62234 | 39032 | 41144 |
| Mw (g/mol) | Peak 2 | 2174 | 1933 | — | 1715 |

Starch branching enzymes can significantly hydrolyze long chain segments of starch, causing a decrease in the overall molecular weight of carriers, increasing the content of α-1,6-glycosidic bonds, and promoting the formation of short chain-rich cluster structures in a sample. Compared to starch, dextrin molecules have inherently damaged structures, and thus are more susceptible to hydrolysis by starch branching enzymes. Peak 1 represents the molecular weight of branched chain polysaccharides, and Peak 2 represents the molecular weight of straight chain polysaccharides. When maltodextrin was used as the substrate, the hydrolysis and transglycosylation of the Ro-GBE were significantly greater than those of the Gt-GBE, and Peak 2 was not detected. It was interesting that the molecular weight of RG-M treated with dual enzymes was between those of Gt-M and Ro-M, which may be due to different optimal chain lengths for the two enzymes. The Gt-GBE tends to relink shorter chain segments (mainly molecules with DP<13), while the Ro-GBE tends to relink longer chain segments (mainly with DP 7-17). Therefore, in RG-M, segments hydrolyzed by dual enzymes can be fully transferred and generate new α-1,6-glycosidic bonds, resulting in an increase in molecular weight.

### Test 4: Determination of Hypocrystalline Layered Structure

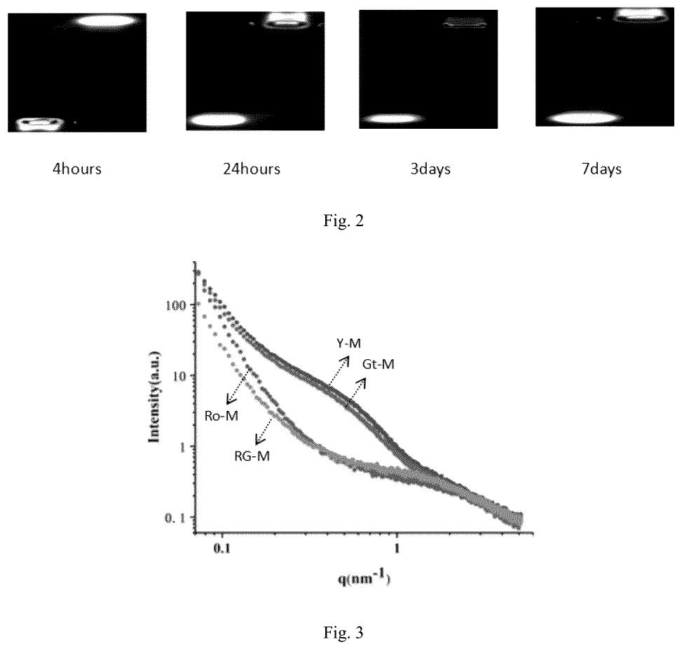

Appropriate amounts of samples (Y-M, Gt-M, Ro-M, RG-M) were weighed separately, prepared into starch milk with a water content of about 60%, and equilibrated at room temperature for 24 h. Then the equilibrated starch milk was pipetted to a capillary tube (2 mm) for testing. Test conditions: copper target Cu-Kα rays with a wavelength of λ=0.15405 nm, 30 min of exposure. The collected signals were homogenized and fitted using SAXS analysis software, and the results are shown in FIG. 3.

According to FIG. 3, a self-similarity structure between geometric order and geometric disorder of aggregates can be analyzed, which has been used for characterizing the structure of starch nanoaggregates. Starch exhibited typical hypocrystalline characteristic scattering peaks around q=0.65-0.67 nm-1. The sample treated with the starch branching enzyme Gt-GBE still contained relatively ordered regions and a small amount of crystalline structure, and retained the original characteristic peaks of starch. The hydrolysis and transglycosylation of the Ro-GBE completely damaged the structure of dextrin, and the newly formed branched dextrin macromolecules had a high degree of disorder.

### Test 5: Determination of Degree of Substitution

The nitrogen content in cationic starch-based gene carriers was determined with reference to GB5009.5-2016, and a calculation formula for the degree of substitution (DS) is as follows: DS=(162×N %)/(1400−151.5×N %).

Table 4 shows the degrees of substitution of the cationic starch-based gene carriers having different degrees of branching prepared by the methods in the aforementioned examples.

TABLE 4
Degrees of substitution of starch-based carriers having different degrees of branching

| Sample | C-RG-S-1 | C-RG-S-4 | C-Y-M-4 | C-Gt-M-4 | C-Ro-M-4 | C-RG-M-1 | C-RG-M-4 |
|---|---|---|---|---|---|---|---|
| Degree of | 0.145 | 0.215 | 0.069 | 0.074 | 0.072 | 0.031 | 0.077 |
| substitution | | | | | | | |

### Example 8

(1) The cationic branched starch-based gene carrier prepared in Example 1 was dissolved in DEPC water to prepare a solution with a concentration of 6.73 mg/ml.

(2) siRNA fragments targeting human gene ABCB1 which encodes P-glycoprotein were synthesized in the laboratory, (the target gene was purchased from Suzhou Genewiz Biotechnology Co., Ltd., China, and the siRNA was synthesized in the laboratory), with a concentration of 2500 ng/μl.

(3) The solutions in step (1) and step (2) were mixed by volume according to the ratios of N/P=0.5, 1.0, 1.5, 2.0, 2.5, 3.0 and 3.5. The siRNA concentration at each N/P was controlled at 2500 ng/μl. An appropriate amount of ultrapure water was added to make the final volume be 10 μl. The mixed solutions were vortexed for 1 min and allowed to stand at room temperature of 25° C. for 1 h.

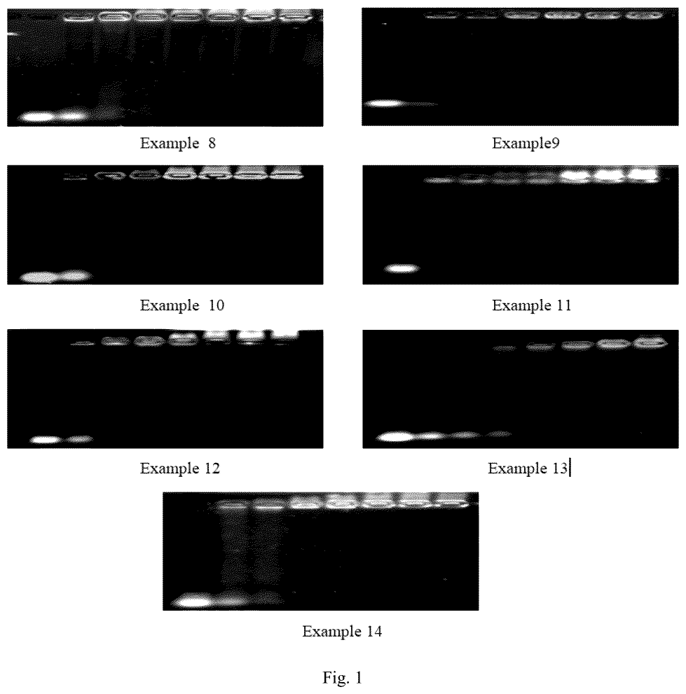

(4) 5 μl of each of the samples with different N/P ratios was mixed with 1 μl of 6× loading buffer and pipetted into 1% agarose wells using a micropipette. A water solution of bare siRNA was used as a control, and electrophoresis was performed in a TAE buffer solution (0.5×) at the voltage of 80 V for 30 min. After electrophoresis, the agarose was transferred into clean distilled water and washed clean, and ultraviolet photos were taken with a gel imager (FIG. 1).

### Example 9

Referring to Example 8, step (1) was changed to: the cationic branched starch-based gene carrier prepared in Example 2 was dissolved in DEPC water to prepare a solution with a concentration of 4.80 mg/ml.

Other conditions were unchanged, and ultraviolet photos were taken with a gel imager (FIG. 1).

### Example 10

Referring to Example 8, step (1) was changed to: the cationic branched starch-based gene carrier prepared in Example 3 was dissolved in DEPC water to prepare a solution with a concentration of 13.25 mg/ml.

Other conditions were unchanged, and ultraviolet photos were taken with a gel imager (FIG. 1).

### Example 11

Referring to Example 8, step (1) was changed to: the cationic branched starch-based gene carrier prepared in Example 4 was dissolved in DEPC water to prepare a solution with a concentration of 12.41 mg/ml.

Other conditions were unchanged, and ultraviolet photos were taken with a gel imager (FIG. 1).

### Example 12

Referring to Example 8, step (1) was changed to: the cationic branched starch-based gene carrier prepared in Example 5 was dissolved in DEPC water to prepare a solution with a concentration of 12.74 mg/ml.

Other conditions were unchanged, and ultraviolet photos were taken with a gel imager (FIG. 1).

### Example 13

Referring to Example 8, step (1) was changed to: the cationic branched starch-based gene carrier prepared in Example 6 was dissolved in DEPC water to prepare a solution with a concentration of 28.53 mg/ml.

Other conditions were unchanged, and ultraviolet photos were taken with a gel imager (FIG. 1).

### Example 14

Referring to Example 8, step (1) was changed to: the cationic branched starch-based gene carrier prepared in Example 7 was dissolved in DEPC water to prepare a solution with a concentration of 11.96 mg/ml.

Other conditions were unchanged, and ultraviolet photos were taken with a gel imager (FIG. 1).

The gel electropherograms of Example 8, 9, 10, 11, 12, 13 and 14 obtained at different N/P ratios are shown in FIG. 1. At a certain N/P ratio, the cationic starch-based gene carriers show good siRNA protection effects. To compare the siRNA loading effects of the cationic gene carriers with the same degree of branching but different degrees of substitution, Examples 8, 9, 13 and 14 in FIG. 1 were compared, and it was found that lower degrees of substitution require higher N/P to achieve better encapsulation. However, with the increase of the mass of the cationic gene carriers, the surface potential of the complexes increases, and the potential biological toxicity increases. Furthermore, to explore the effects of the starch structure on siRNA loading, it is necessary to compare the loading effects of the cationic starch-based gene carriers with similar degrees of substitution but different degrees of branching. Moreover, to obtain carriers with special functional groups to promote the passage of nanoparticles through biological barriers and improve the bioavailability of siRNA, it is necessary to prepare small-sized nanoscale cationic starch-based gene carriers.

### Example 15

The zeta potentials of nanocomplexes formed by the cationic branched dextrin gene carriers prepared in Examples 10, 11, 12 and 14 and siRNA at different N/P was measured using a Malvern laser particle size analyzer. The results show (Table 5) that under the same degree of substitution, regardless of the degrees of branching of the cationic branched dextrin gene carriers, the surface potentials of the complexes formed with the siRNA increased with the increase of the N/P, the charges gradually increased, and the surface potentials were-4.00 to 17.00 mV. The positively charged nanocomplexes can more tightly complex with the negatively charged siRNA, and during transfection, the nanocomplexes can quickly bind to the surface negatively charged cell membranes to promote the endocytosis of cells on the complexes. With the increase of the degree of branching, the surface potentials of the complexes gradually decreased, indicating that a highly branched structure has great potential for reducing the cytotoxicity of the carriers.

Table 5 shows the surface potentials of complexes formed by the cationic branched dextrin gene carriers prepared by the aforementioned methods, having different degrees of branching, and siRNA under different N/P conditions.

TABLE 5
Surface potentials of complexes formed by cationic
branched dextrin gene carriers having different degrees of
branching and siRNA under different N/P conditions

| N/P | C-Y-M-4 | C-Gt-M-4 | C-Ro-M-4 | C-RG-M-4 |
|---|---|---|---|---|
| 0.5 | −3.59 ± 1.77 | −2.47 ± 0.46 | −1.95 ± 0.56 | −2.38 ± 0.27 |
| 1.0 | −0.35 ± 0.15 | 2.15 ± 0.90 | 0.61 ± 0.10 | −1.59 ± 0.35 |
| 1.5 | 4.50 ± 0.13 | 4.38 ± 0.25 | 3.67 ± 0.47 | 4.06 ± 0.38 |
| 2.0 | 10.22 ± 0.92 | 7.92 ± 1.56 | 6.48 ± 0.45 | 6.11 ± 0.03 |
| 2.5 | 10.89 ± 1.69 | 12.50 ± 0.42 | 8.70 ± 0.31 | 8.50 ± 1.99 |
| 3.0 | 13.40 ± 0.46 | 13.55 ± 0.35 | 13.85 ± 0.49 | 11.00 ± 0.28 |
| 3.5 | 17.23 ± 0.06 | 15.30 ± 0.57 | 16.10 ± 1.13 | 13.25 ± 0.21 |

### Example 16

The particle sizes of nanocomplexes formed by the cationic branched dextrin gene carriers prepared in Examples 10, 11, 12 and 14 and siRNA at different N/P were measured using a Malvern laser particle size analyzer. The results show (Table 6) that with the increase of the N/P molar ratio, the particle size first increased and then decreased, which may be due to the fact that when the surface potentials of particles were close to 0 mV, the cationic modified starch, DNA and a delivery system were prone to aggregation, resulting in a significant increase in particle size. With further increase of the N/P, the surfaces became positively charged, and mutual exclusion between particles occurred, so that the particles could exist in the solution in a stable particle size form. At the same degree of substitution, with the increase of the degrees of branching of the cationic branched dextrin gene carriers, the spatial resistance between the complex particles increased. Therefore, under different N/P, the particle size distributions of the complexes formed by cationic hyperbranched dextrin C-RG-M-4 and the siRNA became increasingly uniform, and the resulting nano-delivery systems were 300-400 nm in size.

Table 6 shows the particle size distributions of complexes formed by the cationic branched dextrin gene carriers prepared by the aforementioned methods, having different degrees of branching, and siRNA under different N/P conditions.

TABLE 6
Particle size distributions of complexes formed by cationic branched dextrin gene
carriers having different degrees of branching and siRNA under different N/P conditions

| N/P | C-Y-M-4 | C-Gt-M-4 | C-Ro-M-4 | C-RG-M-4 |
|---|---|---|---|---|
| 0.5 | 659.30 ± 28.99 | 607.55 ± 2.05 | 620.25 ± 14.64 | 312.63 ± 14.47 |
| 1.0 | 635.47 ± 19.80 | 1090.33 ± 63.50 | 509.50 ± 18.53 | 333.83 ± 15.90 |
| 1.5 | 1462.33 ± 25.58 | 513.97 ± 26.97 | 718.25 ± 12.94 | 704.40 ± 22.48 |
| 2.0 | 481.03 ± 19.10 | 394.97 ± 103.74 | 413.75 ± 40.52 | 330.60 ± 7.33 |
| 2.5 | 800.43 ± 59.57 | 382.27 ± 23.95 | 463.80 ± 41.44 | 350.10 ± 20.45 |
| 3.0 | 972.05 ± 100.34 | 553.60 ± 45.16 | 538.03 ± 20.08 | 378.15 ± 2.47 |
| 3.5 | 952.65 ± 81.10 | 710.95 ± 105.00 | 552.15 ± 79.97 | 324.03 ± 13.30 |

### Example 17

The cationic branched dextrin gene carrier solution in Example 14 and an siRNA solution were mixed by volume at the ratio of N/P=2.0. The siRNA concentration was controlled at 2500 ng/μl. An appropriate amount of ultrapure water was added to make the final volume be 10 μl. The mixed solution was vortexed for 1 min and allowed to stand at room temperature of 25° C. for 4 h, 24 h, 3 d and 7 d respectively.

5 μl of each of the samples standing for different times was mixed with 1 μl of 6× loading buffer and pipetted into 1% agarose wells using a micropipette. A water solution of bare siRNA was used as a control, and electrophoresis was performed in a TAE buffer solution (0.5×) at the voltage of 80 V for 30 min. After electrophoresis, the agarose was transferred into clean distilled water and washed clean, and ultraviolet photos were taken with a gel imager (FIG. 2).

Comparing the encapsulation effects of the cationic branched dextrin gene carrier on the siRNA at different times, as shown in FIG. 2, the cationic branched dextrin gene carrier-siRNA complex still showed good encapsulation effects after standing at room temperature for 7 d without leakage of the siRNA.

What is claimed is:

1. A method for preparing a cationic hyperbranched starch-based gene carrier, comprising: using starch or dextrin treated with starch branching enzymes as a substrate, and chemically modifying the substrate with a cationic etherifying agent to obtain a cationic branched starch-based gene carrier, wherein the content of α-1,6-glycosidic bonds in the cationic hyperbranched starch-based gene carrier is 5%-11%; and the degree of substitution of the cationic hyperbranched starch-based gene carrier is 0.030-0.080.

2. The method according to claim 1, wherein specific steps for preparing the cationic hyperbranched starch-based gene carrier are as follows:
(1) preparation of substrate
mixing starch/dextrin with distilled water to prepare a water solution, performing heating and preserving heat for gelatinization, stirring and adding starch branching enzymes for enzymatic hydrolysis, performing gelatinizing and enzyme deactivation after enzymatic hydrolysis, and performing freeze-drying, grinding and screening to obtain the substrate, which is the starch/dextrin treated with the starch branching enzymes; and
(2) preparation of cationic hyperbranched starch-based gene carrier by substrate modification
dispersing the substrate obtained in step (1) in anhydrous ethanol to form a mixture; adjusting the pH of the cationic etherifying agent to 9-10; then adding the cationic etherifying agent to the mixture for reaction by heating; performing cooling to room temperature after the reaction to produce a yellow or light yellow primary product; adding glacial acetic acid to the primary product for neutralization to a neutral pH; and then performing filtering, washing and drying to obtain the cationic hyperbranched starch-based gene carrier.

3. The method according to claim 2, wherein the cationic etherifying agent is a solution of trimethylammonium chloride containing 3-chloro-2-hydroxypropyl.

4. The method according to claim 2, wherein in step (1), the starch branching enzymes are a 1,4-α-glucan branching enzyme from Rhodothermus obamensis (Ro-GBE) and a 1,4-α-glucan branching enzyme from Geobacillus thermoglucosidans (Gt-GBE).

5. The method according to claim 4, wherein in step (1), the starch branching enzyme Gt-GBE has a reaction temperature of 50° C.-60° C., an enzyme concentration of 25-35 U/g, and a reaction time of 10-15 hours.

6. The method according to claim 4, wherein in step (1), the starch branching enzyme Ro-GBE has a reaction temperature of 55° C.-65° C., an enzyme concentration of 30-40 U/g, and a reaction time of 8-12 hours.

7. The method according to claim 2, wherein the prepared cationic hyperbranched starch-based gene carrier is put into a dialysis bag, dialyzed in ultrapure water for 48-72 hours, and then freeze-dried.

8. The method according to claim 1, comprising: using starch or dextrin treated with starch branching enzymes as a substrate, and chemically modifying the substrate with a cationic etherifying agent to obtain a cationic branched starch-based gene carrier, wherein the content of α-1,6-glycosidic bonds in the cationic hyperbranched starch-based gene carrier is 5%-11%; and the degree of substitution of the cationic hyperbranched starch-based gene carrier is 0.030-0.080;
specific steps for preparing the cationic hyperbranched starch-based gene carrier are as follows:
(1) preparation of substrate
mixing starch/dextrin with distilled water to prepare a water solution, performing heating to 60° C. and preserving heat for gelatinization, stirring and adding 35 U/g of a 1,4-α-glucan branching enzyme from Rhodothermus obamensis (Ro-GBE) and 30 U/g of a 1,4-α-glucan branching enzyme from Geobacillus thermoglucosidans (Gt-GBE) for enzymatic hydrolysis for 10 hours, performing gelatinizing and enzyme deactivation after enzymatic hydrolysis, and performing freeze-drying, grinding and screening to obtain the substrate, which is the starch/dextrin treated with the starch branching enzymes; and
(2) preparation of cationic hyperbranched starch-based gene carrier by substrate modification
dispersing the substrate obtained in step (1) in anhydrous ethanol to form a mixture; adjusting the pH of the cationic etherifying agent to 9-10; then adding the cationic etherifying agent to the mixture for reaction by heating; performing cooling to room temperature after the reaction to produce a yellow or light yellow primary product; adding glacial acetic acid to the primary product for neutralization to a neutral pH; and then performing filtering, washing and drying to obtain the cationic hyperbranched starch-based gene carrier.

9. A cationic hyperbranched starch-based gene carrier prepared by the method according to claim 1.

10. Application of the cationic hyperbranched starch-based gene carrier according to claim 9 in preparation of gene drugs.
